(19) United States
(12) Patent Application Publication
(10) Pub. No.: US 2020/0341169 A1
(43) Pub. Date: Oct. 29, 2020

(54) SELF-ADHESIVE SHEET, DENTAL MIRROR, MIRROR FOR INTRAORAL IMAGING AND OPTICAL COMPONENT

(71) Applicant: Sharp Kabushiki Kaisha, Osaka (JP)

(72) Inventor: Kenichiro NAKAMATSU, Sakai City (JP)

(21) Appl. No.: 16/846,814

(22) Filed: Apr. 13, 2020

(30) Foreign Application Priority Data
Apr. 25, 2019 (JP) 2019-084186

(51) Int. Cl. G02B 1/111; G02B 1/18; G02B 1/118; A61B 1/247; C09J 183/04

(52) U.S. Cl. CPC G02B 1/111; G02B 1/18; C09J 183/04; A61B 1/247; G02B 1/118

(57) ABSTRACT

A self-adhesive sheet has a first surface and a second surface which is opposite to the first surface. The first surface includes a plurality of raised portions, a two-dimensional size of the plurality of raised portions being in a range of more than 20 nm and less than 500 nm as viewed from a normal direction of the first surface. The self-adhesive sheet is made of a silicone elastomer having a polydimethyl siloxane backbone, and has a Shore A hardness of not less than 40 and not more than 80. The second surface is capable of pressure-sensitive adhesion.

### BACKGROUND

### 1. Technical Field

The present invention relates to a self-adhesive sheet, and more particularly to a self-adhesive sheet which excels in antireflection property, and a dental mirror, a mirror for intraoral imaging, and an optical component having such a self-adhesive sheet attached on a light-receiving surface thereof.

### 2. Description of the Related Art

Methods that form a minute uneven structure called a moth-eye structure on a surface are being utilized as antireflection techniques. The two-dimensional size of a raised portion of a moth-eye structure is not less than 10 nm but less than 500 nm, for example. Here, the “two-dimensional size” of the raised portions refers to an area equivalent circle diameter of the raised portions viewed in a direction normal to the surface. For example, when the raised portions have a conical shape, the two-dimensional size of the raised portions is equivalent to the diameter of the base of the cone. The same applies to the “two-dimensional size” of the recessed portions.

The present applicant conceived a method for producing an antireflection film (an antireflection surface) which has a moth-eye structure with the use of an anodized porous alumina layer. Using the anodized porous alumina layer enables manufacture of a mold which has an inverted moth-eye structure with high mass producibility (see, for example, Japanese Patent No. 4265729, Japanese Laid-Open Patent Publication No. 2009-166502, WO 2011/125486, and WO 2013/183576). The entire disclosures of Japanese Patent No. 4265729, Japanese Laid-Open Patent Publication No. 2009-166502, WO 2011/125486, and WO 2013/183576 are incorporated by reference in the present specification.

An antireflection coating having the aforementioned moth-eye structure is often formed by using a photocurable resin on a base film, and is attached onto the surface of a display device or an optical element by using an adhesive.

### SUMMARY

However, complicated work is involved in attaching an antireflection coating by using an adhesive. Thus, there exist potential needs for an antireflection coating having self-adhesiveness which is easily removable. Moreover, in some applications that require thermal resistance and/or chemical resistance, no adhesives may be available to be used. For example, for use on a mirror surface such as a dental mirror or a mirror for intraoral imaging, a thermal resistance and/or a chemical resistance for withstanding a sterilization treatment is required.

Therefore, the present invention aims to provide a self-adhesive sheet having an excellent antireflection property which is easily removable, and more particularly a self-adhesive sheet having both thermal resistance and chemical resistance. Moreover, the present invention aims to provide a dental mirror, a mirror for intraoral imaging, and an optical component having such a self-adhesive sheet attached on a light-receiving surface thereof.

According to embodiments of the present invention, solutions as recited in the following Items are provided.

### [Item 1]

A self-adhesive sheet comprising a first surface and a second surface which is opposite to the first surface, wherein,

the first surface includes a plurality of raised portions, a two-dimensional size of the plurality of raised portions being in a range of more than 20 nm and less than 500 nm as viewed from a normal direction of the first surface;

the self-adhesive sheet is made of a silicone elastomer having a polydimethyl siloxane (PDMS) backbone;

the self-adhesive sheet has a Shore A hardness of not less than 40 and not more than 80; and

the second surface is capable of pressure-sensitive adhesion.

More preferably, the Shore A hardness is not less than 60 and not more than 70.

The self-adhesive sheet visible has a light transmittance of e.g. 95% or more.

The self-adhesive sheet has a visible light reflectance of e.g. 0.2% or less.

### [Item 2]

The self-adhesive sheet of Item 1, wherein the silicone elastomer comprises fumed silica.

The proportion of fumed silica contained preferably does not exceed 40 mass % of the entirety. The proportion of fumed silica contained may be 0.1 mass % or less of the entirety. The proportion of fumed silica contained may be e.g. not less than 5 mass % and not more than 30 mass % of the entirety.

### [Item 3]

The self-adhesive sheet of Item 1 or 2, wherein a static contact angle of water with respect to the second surface is 115° or less.

A static contact angle of water with respect to the second surface may be e.g. 95° or less, and may be 80° or less. By irradiating the second surface with excimer UV to modify it, its water contact angle can be reduced.

A static contact angle of hexadecane with respect to the second surface is e.g. 22° or less.

### [Item 4]

The self-adhesive sheet of any of Items 1 to 3, wherein a static contact angle of water with respect to the first surface is 120° or more.

A static contact angle of hexadecane with respect to the first surface is e.g. 40° or less.

### [Item 5]

The self-adhesive sheet of any of Items 1 to 4, wherein the second surface includes a plurality of dented portions, and an area equivalent circle diameter of each of the plurality of dented portions is not less than 1 μm and not more than 50 μm.

Each of the plurality of dented portions is cylindrical, for example.

An adjoining distance (pitch) of the plurality of dented portions may be not less than 1 μm and not more than 100 μm.

### [Item 6]

A dental mirror comprising:

a body including a mirror portion having a mirror surface and a support linked to the mirror portion; and

the self-adhesive sheet of any of Items 1 to 5, wherein,

the second surface of the self-adhesive sheet is adhering to the mirror surface.

The self-adhesive sheet is adhering to the mirror surface so as to be easily removable, for example.

### [Item 7]

A dental mirror comprising:

a body including a mirror portion having a mirror surface and a support linked to the mirror portion; and

the self-adhesive sheet of any of Items 1 to 5, wherein,

the second surface of the self-adhesive sheet is adhesively bonded to the mirror surface via a silicone-based pressure-sensitive adhesive.

### [Item 8]

A mirror for intraoral imaging comprising:

a plate-shaped body including a mirror region having a mirror surface and a support region; and

the self-adhesive sheet of any of Items 1 to 5, wherein,

the second surface of the self-adhesive sheet is adhering to at least a portion of the mirror surface.

### [Item 9]

A mirror for intraoral imaging comprising:

a plate-shaped body including a mirror region having a mirror surface and a support region; and

the self-adhesive sheet of any of Items 1 to 5, wherein,

the second surface of the self-adhesive sheet is adhesively bonded to at least a portion of the mirror surface via a silicone-based pressure-sensitive adhesive.

### [Item 10]

An optical component comprising:

an optical element having a light-receiving surface; and

the self-adhesive sheet of any of Items 1 to 5, wherein,

the second surface of the self-adhesive sheet is adhering to at least a portion of the light-receiving surface.

### [Item 11]

An optical component comprising:

an optical element having a light-receiving surface; and

the self-adhesive sheet of any of Items 1 to 5, wherein,

the second surface of the self-adhesive sheet is adhesively bonded to at least a portion of the light-receiving surface via a silicone-based pressure-sensitive adhesive.

According to an embodiment of the present invention, there is provided a self-adhesive sheet having an excellent antireflection property which is easily removable, and more particularly a self-adhesive sheet having both thermal resistance and chemical resistance. Also according to an embodiment of the present invention, a dental mirror, a mirror for intraoral imaging, and an optical component having such a self-adhesive sheet attached on a light-receiving surface thereof are provided.

### BRIEF DESCRIPTION OF THE DRAWINGS

FIG. 1 is a schematic cross-sectional view showing a self-adhesive sheet 10A according to an embodiment of the present invention.

FIG. 2 is a schematic cross-sectional view showing another self-adhesive sheet 10B according to an embodiment of the present invention.

FIG. 3 is a schematic cross-sectional view of a moth-eye mold 30 used for forming a self-adhesive sheet according to an embodiment of the present invention.

FIG. 4 is a schematic perspective view of a dental mirror 100 including a self-adhesive sheet 10 according to an embodiment of the present invention.

FIG. 5A is a schematic plan view of a mirror for intraoral imaging 200A including a self-adhesive sheet 10a according to an embodiment of the present invention.

FIG. 5B is a schematic plan view of a mirror for intraoral imaging 200B including a self-adhesive sheet 10b according to an embodiment of the present invention.

FIG. 5C is a schematic plan view of a mirror for intraoral imaging 200C including a self-adhesive sheet 10c according to an embodiment of the present invention.

FIG. 5D is a schematic plan view of a mirror for intraoral imaging 200D including a self-adhesive sheet 10d according to an embodiment of the present invention.

FIG. 6A is a schematic side view of a traffic mirror 300 including a self-adhesive sheet 10e according to an embodiment of the present invention.

FIG. 6B is a schematic cross-sectional view of a lighting device 400 of a traffic signal including a self-adhesive sheet 10f according to an embodiment of the present invention.

### DETAILED DESCRIPTION

Hereinafter, with reference to the drawings, a self-adhesive sheet and a dental mirror according to an embodiment of the present invention will be described. Note that embodiments of the present invention are not limited to the embodiments illustrated below.

FIG. 1 shows a schematic cross-sectional view of a self-adhesive sheet 10A according to an embodiment of the present invention. The self-adhesive sheet 10A is made of a silicone elastomer 12 having a polydimethyl siloxane backbone, and has a Shore A hardness of not less than 40 and not more than 80. From a viewpoint of strength, the silicone elastomer 12 preferably contains fumed silica. The proportion of fumed silica contained preferably does not exceed 40 mass % of the entirety. The proportion of fumed silica contained may be 0.1 mass % or less of the entirety. For example, the proportion of fumed silica contained may be e.g. not less than 5 mass % and not more than 30 mass % of the entirety. The fumed silica preferably is silica particles having an average particle size of 50 nm or less, and in view of the influences of scatter by moth-eye shaped particles, the average particle size is more preferably 30 nm or less.

As will be described later through experimental examples, a silicone elastomer having a Shore A hardness of not less than 40 and not more than 80 has moderate flexibility and strength, and therefore may possess self-adhesiveness. Moreover, as is well known, a silicone elastomer has excellent thermal resistance and chemical resistance. Therefore, when used in a dental mirror to be described latter, too, it can endure a sterilization treatment (for example, cleaning with a neutral detergent and/or a heat treatment at e.g. about 130° C.)

The self-adhesive sheet 10A has a plurality of raised portions 12p on a first surface 12S. The plurality of raised portions 12p constitute a moth-eye structure. When viewed in a normal direction of the self-adhesive sheet 10A, the two-dimensional size of the raised portions 12p, Dₚ, is in the range of more than 20 nm and less than 500 nm. Here, the “two-dimensional size” of the raised portions 12p refers to the diameter of a circle equivalent to the area of the raised portions 12p when viewed in a normal direction of the surface. When the raised portions 12p have a conical shape, for example, the two-dimensional size of the raised portions 12p is equivalent to the diameter of the base of the cone. The typical adjoining distance of the raised portions 12p, Dint, is more than 20 nm but not more than 1000 nm. When the raised portions 12p are densely arranged so that there is no gap between adjoining raised portions 12p (e.g., the bases of the cones partially overlap each other) as shown in FIG. 1, the two-dimensional size of the raised portions 12p, Dₚ, is equal to the adjoining distance Dint. The typical height of the raised portions 12p, Dₕ, is not less than 50 nm but less than 500 nm. The height Dₕ of the raised portions 12p may be not more than 150 nm. The thickness of the self-adhesive sheet 10A is not particularly limited; for example, it may be not less than 100 μm and not more than 2000 μm from the viewpoint of strength and handling ease.

The self-adhesive sheet 10A has the same moth-eye structure as the antireflection films disclosed in Japanese Patent No. 4265729, Japanese Laid-Open Patent Publication No. 2009-166502, WO 2011/125486 and WO 2013/183576. From the viewpoint of producing an antireflection function, it is preferred that the surface has no flat portion, and the raised portions 12p are densely arranged over the surface. Further, the raised portions 12p preferably has a such shape that the cross-sectional area (a cross section parallel to a plane which is orthogonal to an incoming light ray) increases from the air side to the inside, e.g., a conical shape. From the viewpoint of suppressing interference of light, it is preferred that the raised portions 12p are arranged without regularity, preferably randomly. The visible light reflectance of the self-adhesive sheet 10A may be e.g. 0.2% or less, and the visible light transmittance may be e.g. 95% or more.

Moreover, the moth-eye structure which the first surface 12S possesses exhibits so-called Lotus effect, and thus the first surface 12S exhibits excellent water repellency. A static contact angle of water with respect to the first surface 12S may be e.g. 120° or more. On the other hand, a second surface 14A which is on the opposite side to the first surface 12S is flat, and a static contact angle of water with respect to the second surface 14A is 115° or less. By irradiating the second surface with excimer UV (wavelength: 172 nm) to modify it, the water contact angle can be reduced. A static contact angle of water with respect to the second surface 14A may be e.g. 95° or less, and may be 80° or less. The smaller the water contact angle with respect to the second surface 14A is, i.e., the more hydrophilic the second surface is, the more adhesive the second surface becomes.

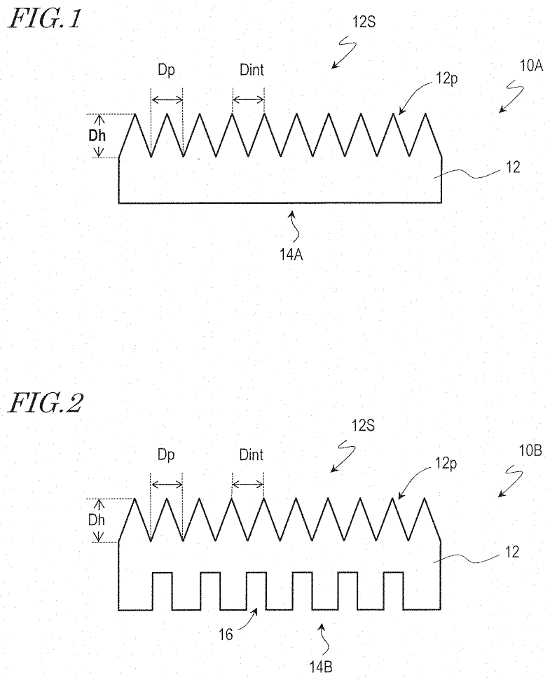

FIG. 2 shows a schematic cross-sectional view of another self-adhesive sheet 10B according to an embodiment of the present invention. A first surface 12S of the self-adhesive sheet 10B may have the same configuration as that of the first surface 12S of the self-adhesive sheet 10A. The self-adhesive sheet 10B differs from the self-adhesive sheet 10A in that the second surface 14B includes a plurality of dented portions 16.

The plurality of dented portions 16 of the second surface 14B each have an area equivalent circle diameter of e.g. not less than 1 μm and not more than 50 μm. The plurality of dented portions 16 may each be cylindrical, for example. Moreover, an adjoining distance (pitch) of the plurality of dented portions is not less than 1 μm and not more than 100 μm. Note that the plurality of dented portions do not need to be regularly arranged. Molds having microscopic raised portions (e.g., circular cylinders) corresponding to such dented portions 16 are commercially available.

After the second surface 14B of the self-adhesive sheet 10B is placed in contact with a surface to which it is expected to adhere (e.g., a mirror surface of a dental mirror), as the self-adhesive sheet 10B is pressed from the first surface 12S, air within the plurality of dented portions 16 is pushed out, the plurality of dented portions 16 act as suckers, thereby further enhancing adhesion of the second surface 14. Once the air within the plurality of dented portions 16 is pushed out, reflection on the second surface 14 is suppressed, whereby the self-adhesive sheet 10B appears more transparent.

In the present specification, a “moth-eye structure” is constituted by raised portions each having a shape such that its cross-sectional area (i.e., a cross section that is parallel to the film surface) increases as that of the raised portions 12p on the first surfaces 12S of self—the adhesive sheet 10A and 10B shown in FIG. 1 and FIG. 2. When the raised portions 12p have a conical shape, the slope appearing in their cross section will be a straight line; however, this is not a limitation, and this may alternatively a curve.

FIG. 3 shows a schematic cross-sectional view of a mold for forming a moth-eye structure such as illustrated in FIG. 1 and FIG. 2 over the surface (hereinafter, referred to as “moth-eye mold”). On its surface, the moth-eye mold 30 has an inverted moth-eye structure obtained by inverting the moth-eye structure. Such a moth-eye mold 30 can be manufactured according to methods disclosed in Japanese Laid-Open Patent Publication No. 2009-166502, WO 2011/125486, and WO 2013/183576. That is, by alternately and repeatedly performing the anodization step and the etching step on an aluminum film deposited on a base or on an aluminum base through multiple cycles, a moth-eye mold is obtained which includes a porous alumina layer which has an inverted moth-eye structure.

A porous alumina layer 34 (having a thickness tₚ) shown in FIG. 3 includes a porous layer (having a thickness corresponding to the depth D_{d} of dented portions 34p) and a barrier layer (having a thickness t_{b}). Since the porous alumina layer 34 has a structure obtained by inverting the moth-eye structure of the self-adhesive sheets 10A and 10B, corresponding parameters which define the dimensions may sometimes be designated by the same symbols.

The dented portions 34p of the porous alumina layer 34 may each have a conical shape, for example, and have a stepped side surface. Preferably, the two-dimensional size Dₚ of the dented portions 34p (i.e., an area equivalent circle diameter of the dented portion as viewed from the surface normal) is more than 20 nm and less than 500 nm, and the depth D_{d} is about not less than 50 nm but less than 1000 nm (1 μm). Moreover, the bottom of each dented portion 34p is pointed (i.e., the bottommost portion substantially defines a point). In the case where the dented portions 34p are closely-packed, assuming that the shape of each dented portion 34p as viewed from the normal direction of the porous alumina layer 34 is a circle, adjacent circles overlap each other, and a saddle portion is created between adjacent dented portions 34p. In the case where dented portions 34p of substantially conical shapes are adjacent to each other so as to create a saddle portion in between, the two-dimensional size Dₚ of each dented portion 34p is equal to the adjoining distance Dint. The thickness tₚ of the porous alumina layer 34 may be about 1 μm or less, for example.

Note that, under the porous alumina layer 34 shown in FIG. 3, a residual aluminum layer 38r, which is a portion of the aluminum film 38 that was not anodized, exists. As necessary, the aluminum film 38 may be substantially completely anodized so that no residual aluminum layer 38r will exist. For example, in the case where the inorganic material layer 36 is thin, an electric current can be easily supplied from the aluminum base 32 side.

The self-adhesive sheet 10A is obtained by applying a silicone elastomer precursor between the moth-eye mold shown in FIG. 3 and a flat-plate mold, and curing the precursor through heating, for example. Moreover, the self-adhesive sheet 10B is obtained by applying a silicone elastomer precursor between the moth-eye mold shown in FIG. 3 and a mold having microscopic raised portions (e.g., circular cylinders) corresponding to such dented portions 16, and curing the precursor through heating, for example.

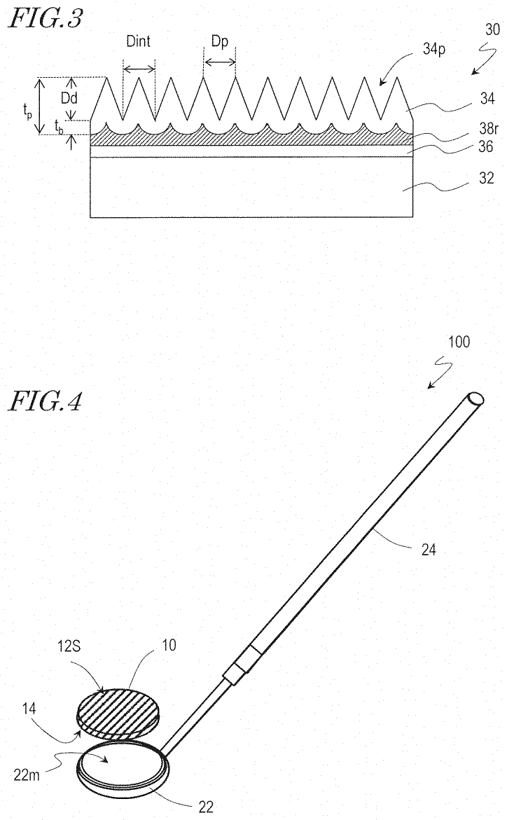

The self-adhesive sheet 10A and 10B may be used by being attached onto a mirror surface of a dental mirror. FIG. 4 shows a schematic perspective view of a dental mirror 100 including the self-adhesive sheet 10 according to an embodiment of the present invention.

The dental mirror 100 includes a mirror portion 22 having a mirror surface 22m and a support 24 that is linked to the mirror portion 22, where the second surface 14 of the self-adhesive sheet 10 is adhering to the mirror surface 22m. The self-adhesive sheet 10 may be adhering to the mirror surface 22m in a manner which permits easy removal, for example. Note that the self-adhesive sheet 10 may be firmly attached onto the mirror surface 22m through heating (e.g. at 150° C. or above). At this time, the second surface 14 of the self-adhesive sheet 10 may be made hydrophilic by being irradiated with excimer UV (172 nm), for example.

Hereinafter, some experimental examples will be shown to illustrate the characteristics of the self-adhesive sheet according to according to an embodiment of the present invention.

As the materials of a silicone elastomer having a polydimethyl siloxane backbone, Sylgard 184 (manufactured by Dow Corning Corp.; Sylgard is a registered trademark) and LSR series (manufactured by Momentive) were used. Sylgard 184 contains fumed silica in an amount of not less than 10 mass % and not more than 30 mass % based on the entirety. LSR series contains fumed silica in an amount of 0.1 mass % or less based on the entirety.

Table 1 shows results of evaluating the Shore A hardness, frictional force (or friction resistance), contact angles (water, hexadecane) of each silicone elastomer sheet (flat surface). The surface of each silicone elastomer sheet was modified through excimer UV irradiation for similar evaluations. None of the sheets showed any deterioration through excimer UV irradiation.

TABLE 1

| silicone | | | | | water contact angle | | hexadecane contact | |
|---|---|---|---|---|---|---|---|---|
| elastomer | excimer | hard- | frictional force (N) | | (°) | | angle (°) | |
| (flat | UV irra- | ness | 0.5 H after | 24 H after | 0.5 H after | 24 H after | 0.5 H after | 24 H after |
| surface) | diation | Shore A | irradiation | irradiation | irradiation | irradiation | irradiation | irradiation |
| Sylgard184 | — | 43 | 1.0-1.1 | 1.0-1.1 | 113.5 | 113.7 | 20.0 | 20.0 |
| | 50 mW, | | 2.6-2.7 | 2.7-2.8 | 63.2 | 92.7 | <10. | 12.8 |
| | 7 min. | | | | | | | |
| TSR7060 | — | 60 | 1.0-1.2 | 1.0-1.2 | 105.2 | 105.2 | 20.5 | 20.5 |
| | 50 mW, | | 2.9-3.0 | 2.5-2.6 | 57.8 | 74.7 | <10. | <10. |
| | 7 min. | | | | | | | |
| TSR7070 | — | 70 | 1.0-1.1 | 1.0-1.1 | 103.8 | 103.8 | 21.8 | 21.5 |
| | 50 mW, | | 2.5-2.6 | 2.4-2.5 | 48.3 | 66.7 | <10. | <10. |
| | 7 min. | | | | | | | |
| TSR7080 | — | 80 | 0.7-0.8 | 0.7-0.8 | 101.3 | 101.3 | 19.7 | 19.5 |
| | 50 mW, | | 2.7-2.8 | 2.3-2.4 | 12.6 | 51.5 | <10. | <10. |
| | 7 min. | | | | | | | |
| TSR7090 | — | 90 | 0.6-0.7 | 0.6-0.7 | 103.5 | 103.5 | 20.2 | 20.8 |
| | 50 mW, | | 1.8-1.9 | 1.5-1.6 | 9.1 | 43.4 | <10. | <10. |
| | 7 min. | | | | | | | |

The frictional force was evaluated by, with a nonwoven fabric (Savina) having a smooth surface, rubbing the surface of each silicone elastomer sheet once with a load of 100 g and at a speed of 30 mm/min. Using a contact angle meter (manufactured by Kyowa Interface Science, Inc.; PCA-1), static contact angles of water and hexadecane of each sample film with respect to the surface of the synthetic polymer film were measured.

Each silicone elastomer sheet was allowed to adhere to a flat surface (e.g., of an acrylic plate), and the strength and workability of the sheet were evaluated. While every sheet proved adhesive, those sheets having a Shore A hardness of 90 were found to have high strength but inferior flexibility, thus hindering adhesion. Sylgard 184, having a Shore A hardness of 43, had somewhat lower strength, and may have become ripped. In applications based on its self-adhesiveness, e.g. dental mirrors, it might suffice for disposable use dedicated to each patient, but would slightly lack in strength for repeated usage. Sheets having a Shore A hardness of 80 also slightly lacked in flexibility. Sheets having a Shore A hardness of 60 or 70 had the best balance between flexibility and strength.

After a surface modification through excimer UV irradiation, each sheet had a reduced water contact angle, indicative of improved hydrophilicity. Frictional force is also considered to have improved consequently.

Table 2 shows results of evaluating the moth-eye surface of each silicone elastomer sheet.

TABLE 2

| silicone | | | water | hexadecane |
|---|---|---|---|---|
| elastomer | | frictional | contact | contact |
| (moth-eye | hardness | force | angle | angle |
| surface) | Shore A | (N) | (°) | (°) |
| Sylgard184 | 43 | 0.8 | 120.5 | 28.5 |
| LSR7060 | 60 | 0.6 | 122.5 | 35.1 |
| LSR7070 | 70 | 0.6 | 124.8 | 32.8 |
| LSR7080 | 80 | 0.6 | 122.3 | 35.1 |
| LSR7090 | 90 | 0.6 | 121.5 | 33.7 |

As can be seen from a comparison between Table 2 and Table 1, the contact angle was greater for the moth-eye surface than for the flat surface, both with respect to water and hexadecane. This is ascribable to the Lotus effect provided by the moth-eye surface.

The moth-eye surface having excellent water repellency (having a water static contact angle of 120° or more) also had an anti-fogging effect. As shown in FIG. 4, when the self-adhesive sheet was attached to a mirror surface of a dental mirror, the sheet surface showed no fogging during intraoral observation.

Moreover, the moth-eye surface has an excellent antireflection property, and provides a reflectance of 0.2% or less across the entire spectrum of visible light (380 nm to 780 nm). Moreover, the reflectance of 0.2% or less is available in a wide range of incident angles. Therefore, with a dental mirror including an self-adhesive sheet according to an embodiment of the present invention, a vivid reflection image with high color reproducibility can be obtained.

By using a silane coupling agent, the first surface or the second surface of the self-adhesive sheet may be modified so as to become hydrophilic or hydrophobic (water repellent). For example, the first surface may be modified to become water repellent, while the second surface may be further modified to become hydrophilic. It would be preferable to radiate excimer UV so as to once modify the surface to become hydrophilic and then treat it with a silane coupling agent, because this will allow the silane coupling agent to hydrolyze and thereafter effectively couple to the surface of the self-adhesive sheet.

The self-adhesive sheet according to an embodiment of the present invention is (or rather, its second surface is) in itself self-adhesive, and therefore may be directly attached onto a mirror surface of a dental mirror for use as described above. However, a pressure-sensitive adhesive may additionally be used for its attachment. However, it was found that, in some cases, using e.g. an acrylic pressure-sensitive adhesive (for example, PD-S1 manufactured by PANAC CO., LTD.) as a pressure-sensitive adhesive to attach the self-adhesive sheet onto a mirror surface of a dental mirror, followed by a sterilization treatment (e.g., after cleaning with a neutral detergent, heating in an autoclave at 135° C.), would deteriorate the pressure-sensitive adhesive, such that it could not be thoroughly removed off the mirror surface (thus resulting in some residual islets of pressure-sensitive adhesive).

On the other hand, by using a silicone-based pressure-sensitive adhesive having a PDMS backbone (e.g., SI75-100 manufactured by Nicheikako Co., Ltd.) as the pressure-sensitive adhesive, the pressure-sensitive adhesive will not deteriorate even after a sterilization treatment, so that it can be thoroughly removed off the mirror surface. Therefore, in the case of using a pressure-sensitive adhesive in order to realize firmer adhesive bonding, for example, it is preferable to use a silicone-based pressure-sensitive adhesive having a PDMS backbone.

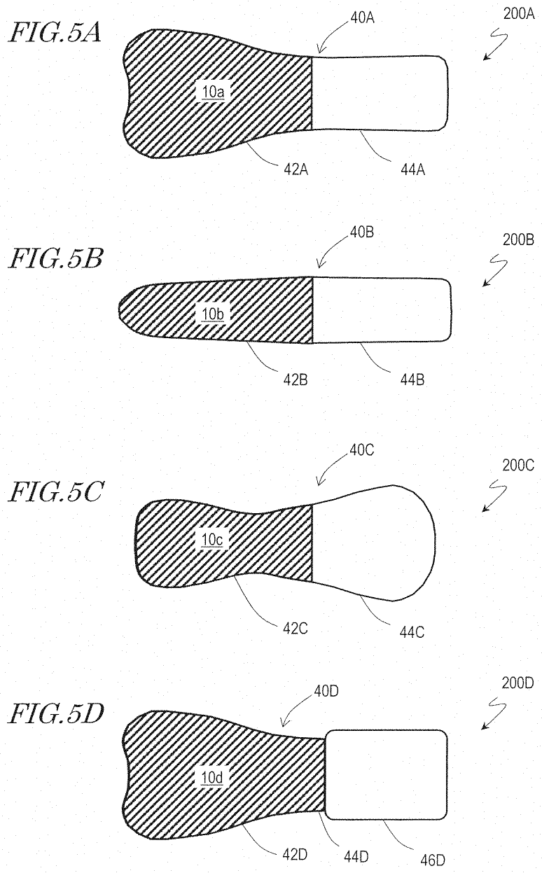

A self-adhesive sheet according to an embodiment of the present invention is also suitably used for a mirror for intraoral imaging, as well as for a dental mirror. FIGS. 5A through 5D show schematic plan views of mirrors for intraoral imaging 200A through 200D, which respectively include self-adhesive sheets 10a through 10d according to embodiments of the present invention.

As shown in FIG. 5A, the mirror for intraoral imaging 200A includes: a plate-shaped body 40A that includes a mirror region 42A having a mirror surface and a support region 44; and a self-adhesive sheet 10a. The adhesive second surface of the self-adhesive sheet 10a is adhering to at least a portion of the mirror surface. The body 40A is made of e.g. a stainless steel, and the mirror region 42A has a rhodium coating thereon, for example. The entire body 40A may define the mirror region 42A.

Mirrors for intraoral imaging are commercially available in various shapes and sizes. Now the examples of FIG. 5B, FIG. 5C, and FIG. 5D will be described.

The mirror for intraoral imaging 200B shown in FIG. 5B includes: a plate-shaped body 40B that includes a mirror region 42B having a mirror surface and a support region 44B; and a self-adhesive sheet 10b. The adhesive second surface of the self-adhesive sheet 10b is adhering to at least a portion of the mirror surface. The mirror for intraoral imaging 200B is for use at the lateral sides, and thus the body 40B has an elongated shape.

The mirror for intraoral imaging 200C shown in FIG. 5C also includes: a plate-shaped body 40C that includes a mirror region 42C having a mirror surface and a support region 44C; and a self-adhesive sheet 10c. The adhesive second surface of the self-adhesive sheet 10c is adhering to at least a portion of the mirror surface. The mirror for intraoral imaging 200C may be used for smaller mouths than the mirror for intraoral imaging 200A may be.

The mirror for intraoral imaging 200D shown in FIG. 5D includes: a plate-shaped body 40D that includes a mirror region 42D having a mirror surface and a support region 44D; and a self-adhesive sheet 10d. The adhesive second surface of the self-adhesive sheet 10d is adhering to at least a portion of the mirror surface. The mirror for intraoral imaging 200D includes a grip 46D covering an end of the support region 44D, thus resulting in a configuration that is easier to hold than the mirror for intraoral imaging 200A.

Without being limited to the above examples, self-adhesive sheets according to embodiments of the present invention can be used for various optical elements having a light-receiving surface of which an antireflection property is expected. In particular, a self-adhesive sheet having excellent water repellency is for outdoor use. For example, the adhesive surface of a self-adhesive sheet may be adhering to at least a portion of the light-receiving surface of any of various optical elements, e.g., a lens of a security camera, a traffic mirror (curve mirror), lighting devices of a traffic signal. A self-adhesive sheet having excellent water repellency can exhibit not only an anti-fogging property but also an anti-smear property, and therefore is suitable for outdoor applications.

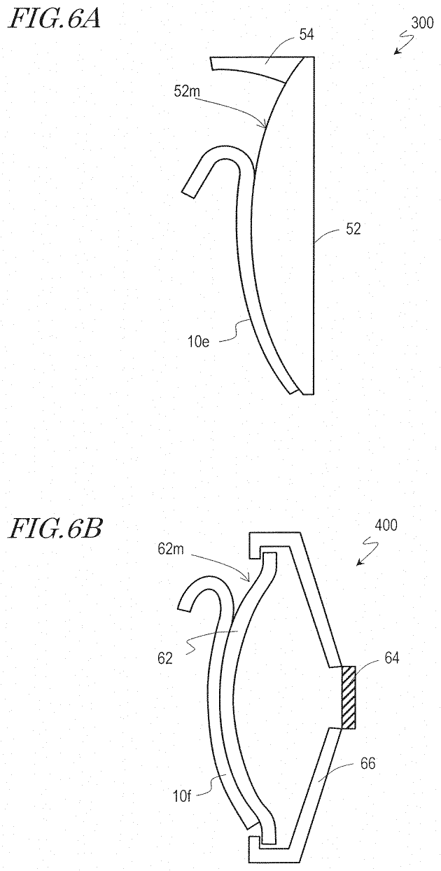

FIG. 6A shows a schematic side view of a traffic mirror 300 including a self-adhesive sheet 10e according to an embodiment of the present invention. The traffic mirror 300 includes a reflector body 52, a hood 54, and a self-adhesive sheet 10e. The adhesive second surface of the self-adhesive sheet 10e is adhering to a mirror surface 52m of the reflector body 52. By using a self-adhesive sheet whose first surface has excellent water repellency (having a water static contact angle of 120° or more) as the self-adhesive sheet 10e, an excellent anti-fogging property and/or anti-smear property will be obtained, whereby the mirror surface 52m can be kept clean. Furthermore, since self-adhesiveness is utilized, as schematically shown in FIG. 6A, it can be easily removed off the mirror surface 52m, thus providing an advantage of easy maintenance work.

FIG. 6B shows a schematic cross-sectional view of a lighting device 400 of a traffic signal including a self-adhesive sheet 10f according to an embodiment of the present invention. The lighting device 400 includes an external lens 62, a light source (e.g., an LED) 64, a housing 66, and a self-adhesive sheet 10f. Although the device 400 may further include an internal lens and the like, any such elements are omitted for simplicity. The adhesive second surface of the self-adhesive sheet 10f is adhering to a light-receiving surface 62m of the external lens 62.

Since the first surface of the self-adhesive sheet 10f has an excellent antireflection property, reflection of light that is externally incident on the light-receiving surface 62m of the external lens 62 is suppressed, whereby any light outgoing from the lighting device can be restrained from becoming obscured by reflected light. Furthermore, as in the aforementioned use with the traffic mirror 300, an excellent anti-fogging property and an excellent anti-smear property can be conferred by the use of the self-adhesive sheet 10f whose first surface has excellent water repellency. Furthermore, since self-adhesiveness is utilized, as schematically shown in FIG. 6B, removal off the light-receiving surface 62m is easy, thus providing an advantage of easy maintenance work.

Not only in dental mirrors as has been mentioned above, but also in other applications, a self-adhesive sheet according to an embodiment of the present invention may be attached onto a light-receiving surface via a pressure-sensitive adhesive. In this case, a preferable pressure-sensitive adhesive to be used is a silicone-based pressure-sensitive adhesive having a PDMS backbone.

As described above, a self-adhesive sheet according to an embodiment of the present invention can adhere, at its adhesive second surface, to the mirror surface of a dental mirror (e.g., glass surface), the surface of a mirror for intraoral imaging (e.g. the surface of a rhodium film on a stainless steel), the mirror surface of a traffic mirror (e.g., the surface of a stainless steel, an acrylic resin, a polycarbonate resin, or glass), the light-receiving surface (glass surface) of an external lens of a lighting device of a traffic signal, or the like. The surface to which the self-adhesive sheet (second surface) is adhering may be made of either a metal material, an inorganic material (glass), or a resin material, and may be a plane or a curved surface. However, the surface to which the self-adhesive sheet is adhering is preferably smooth, and preferably has high hydrophilicity. In the case of attaching the self-adhesive sheet via a pressure-sensitive adhesive, the surface smoothness may be low.

A self-adhesive sheet according to an embodiment of the present invention is suitably used for a dental mirror, a mirror for intraoral imaging, and various other optical components.

This application is based on Japanese Patent Applications No. 2019-084186 filed on Apr. 25, 2019, the entire content of which is hereby incorporated by reference.

What is claimed is:

1. A self-adhesive sheet comprising a first surface and a second surface which is opposite to the first surface, wherein,
the first surface includes a plurality of raised portions, a two-dimensional size of the plurality of raised portions being in a range of more than 20 nm and less than 500 nm as viewed from a normal direction of the first surface;
the self-adhesive sheet is made of a silicone elastomer having a polydimethyl siloxane backbone;
the self-adhesive sheet has a Shore A hardness of not less than 40 and not more than 80; and
the second surface is capable of pressure-sensitive adhesion.

2. The self-adhesive sheet of claim 1, wherein the silicone elastomer comprises fumed silica.

3. The self-adhesive sheet of claim 1, wherein a static contact angle of water with respect to the second surface is 115° or less.

4. The self-adhesive sheet of claim 1, wherein a static contact angle of water with respect to the first surface is 120° or more.

5. The self-adhesive sheet of claim 1, wherein the second surface includes a plurality of dented portions, and an area equivalent circle diameter of each of the plurality of dented portions is not less than 1 μm and not more than 50 μm.

6. A dental mirror comprising:
a body including a mirror portion having a mirror surface and a support linked to the mirror portion; and
the self-adhesive sheet of claim 1, wherein,
the second surface of the self-adhesive sheet is adhering to the mirror surface.

7. A dental mirror comprising:
a body including a mirror portion having a mirror surface and a support linked to the mirror portion; and
the self-adhesive sheet of claim 1, wherein,
the second surface of the self-adhesive sheet is adhesively bonded to the mirror surface via a silicone-based pressure-sensitive adhesive.

8. A mirror for intraoral imaging comprising:
a plate-shaped body including a mirror region having a mirror surface and a support region; and
the self-adhesive sheet of claim 1, wherein,
the second surface of the self-adhesive sheet is adhering to at least a portion of the mirror surface.

9. A mirror for intraoral imaging comprising:
a plate-shaped body including a mirror region having a mirror surface and a support region; and
the self-adhesive sheet of claim 1, wherein,
the second surface of the self-adhesive sheet is adhesively bonded to at least a portion of the mirror surface via a silicone-based pressure-sensitive adhesive.

10. An optical component comprising:
an optical element having a light-receiving surface; and
the self-adhesive sheet of claim 1, wherein,
the second surface of the self-adhesive sheet is adhering to at least a portion of the light-receiving surface.

11. An optical component comprising:
an optical element having a light-receiving surface; and
the self-adhesive sheet of claim 1, wherein,
the second surface of the self-adhesive sheet is adhesively bonded to at least a portion of the light-receiving surface via a silicone-based pressure-sensitive adhesive.
